(19) United States
(12) Patent Application Publication
(10) Pub. No.: US 2022/0195483 A1
(43) Pub. Date: Jun. 23, 2022

(54) DIAGNOSING HEART DISEASE AND DEGENERATIVE MITRAL VALVE DISEASE IN A CANINE

(71) Applicant: Société des Produits Nestlé S.A., Vevey (CH)

(72) Inventor: Qinghong Li, Chesterfield, MO (US)

(21) Appl. No.: 17/547,845

(22) Filed: Dec. 10, 2021

Related U.S. Application Data
(60) Provisional application No. 63/127,247, filed on Dec. 18, 2020.

(51) Int. Cl. C12Q 1/06; C12Q 1/14

(52) U.S. Cl. CPC C12Q 1/06; C12Q 1/14

(57) ABSTRACT

The present invention relates to methods for diagnosing heart disease in a canine, including early stage degenerative mitral valve disease, by using microbiome including specific genera and species. In one embodiment, the method can comprise measuring a normalized relative abundance of fecal bacteria including Faecalibacterium, Turicibacter, Streptococcus, E. Coli, Blautia, Fusobacterium, and C. hiranonis, calculating a dysbiosis index based on the fecal bacteria, and determining that the canine has heart disease if the dysbiosis index is greater than −1.0.

### CROSS REFERENCE TO RELATED APPLICATION

This application claims priority to U.S. Provisional Application Ser. No. 63/127,247 filed Dec. 18, 2020, the disclosure of which is incorporated in its entirety herein by this reference.

### BACKGROUND

Canine degenerative mitral valve disease (DMVD) is characterized by slowly progressive valvular degeneration that causes mitral regurgitation and, in some dogs, congestive heart failure (CHF). Although dogs in the early stage typically have a lengthy preclinical period, once progressed to the stage with CHF, the disease advances more rapidly with a mean survival time less than 12 months. Thus, it is of great interest to intervene at the early preclinical stage to extend the longevity of affected dogs. In recent years, a staging scheme for classifying canine DMVD has been adopted by the consensus committee established by the American College of Veterinary Internal Medicine (ACVIM). Dogs at risk of developing DMVD but otherwise healthy are considered stage A; dogs with a heart murmur due to mitral regurgitation but without clinical signs of CHF are classified as stage B; dogs with overt clinical signs of CHF are classified as stage C. Stage B dogs are further divided into stage B1 or B2 due to the absence or presence of cardiac remodeling.

Currently, the only medication that has been proven to be effective for early preclinical DMVD is pimobanden, which, like any pharmaceutical drug, comes with side effects. Currently, the gold standard for DMVD diagnosis is echocardiogram, which is not only expensive but also requires highly specialized veterinary cardiologist. As such, effective diagnostic methods and treatments overcoming the disadvantages of current methods and treatments continue to be sought.

### SUMMARY

The present disclosure relates generally to diagnosing heart disease and degenerative mitral valve disease (DMVD) in a canine, and in one aspect, early stage DMVD. In one embodiment, a method of diagnosing heart disease in a canine can comprise measuring a normalized relative abundance of fecal bacteria including Faecalibacterium, Turicibacter, Streptococcus, E. Coli, Blautia, Fusobacterium, and C. hiranonis, calculating a dysbiosis index based on the fecal bacteria, and determining that the canine has heart disease if the dysbiosis index is greater than −1.0.

In another embodiment, a method of diagnosing early stage DMVD in a canine can comprise measuring a normalized relative abundance of a biomarker selected from the group consisting of Catenibacterium mitsuokai, Butyricicoccus pullicaecorum, Bacteroides coprocola, Bacteroides plebeius, Allobaculum stercoricanis, or combinations thereof; and determining that the canine has early stage DMVD if the normalized relative abundance of Catenibacterium mitsuokai is between 0.3 to 3, the normalized relative abundance of Butyricicoccus pullicaecorum is between 0.14 to 0.35, the normalized relative abundance of Bacteroides coprocola is between 0.6 to 1.3, the normalized relative abundance of Allobaculum stercoricanisi is between 0.1 and 1.5.

In yet another embodiment, a method of diagnosing early stage DMVD in a canine can comprise measuring a normalized relative abundance of bacteria in a genus, wherein the genus is selected from the group consisting of Catenibacterium, Prevotella, Butyricicoccus, Faecalibacterium, Clostridium, Allobaculum, or combinations thereof; and determining that the canine has early stage DMVD if the normalized relative abundance of the bacteria in the Catenibacterium genus is between 0.3 to 3, the normalized relative abundance of the bacteria in the Prevotella genus is between 0.5 to 4, the normalized relative abundance of the bacteria in the Butyricicoccus genus is between 0.14 to 0.4, the normalized relative abundance of the bacteria in the Faecalibacterium genus is between 0.012 to 0.04, the normalized relative abundance of the bacteria in the Clostridium genus is between 2 and 4, or the normalized relative abundance of the bacteria in the Allobaculum genus is between 0.2 and 1.5.

Additional features and advantages are described herein and will be apparent from the following Detailed Description

### DETAILED DESCRIPTION

Definitions

As used in this disclosure and the appended claims, the singular forms “a,” “an” and “the” include plural referents unless the context clearly dictates otherwise. Thus, for example, reference to “a fecal bacteria or bacterium” or “the fecal bacteria or bacterium” includes two or more such bacteria or bacterium. The term “and/or” used in the context of “X and/or Y” should be interpreted as “X,” or “Y,” or “X and Y.” Where used herein, the terms “example” and “such as,” particularly when followed by a listing of terms, are merely exemplary and illustrative, and are not exclusive or comprehensive.

As used herein, “about” is understood to refer to numbers in a range of numerals, for example the range of −10% to +10% of the referenced number, within −5% to +5% of the referenced number, or in one aspect, within −1% to +1% of the referenced number, and in a specific aspect, within −0.1% to +0.1% of the referenced number. Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used herein, “between” is inclusive of the endpoints. For example, a dysbiosis index between −1 and 0.5 includes where the dysbiosis is −1 or 0.5.

All percentages expressed herein are by weight of the composition on a dry matter basis unless specifically stated otherwise. The skilled artisan will appreciate that the term “dry matter basis” means that an ingredient's concentration or percentage in a composition is measured or determined after any free moisture in the composition has been removed. When reference is made to the pH, values correspond to pH measured at 25° C. with standard equipment. An “amount” can be the total amount of the referenced component per serving of the composition or per distinct unit of the composition and/or can be the weight percentage of the referenced component by dry weight. Moreover, an “amount” includes zero; for example, the recitation of an amount of a compound does not necessarily mean that the compound is present, unless followed by a range that excludes zero.

As used herein, “normalized relative abundance” refers to the amount of each microorganism calculated by taking each count and dividing by the total sequence count in each sample and transforming by square root.

As used herein, “early stage degenerative mitral valve disease” refers to stage B of degenerative mitral valve disease.

As used herein, “stage A” refers to dogs that are at risk of developing degenerative mitral valve disease, but otherwise have a healthy heart.

As used herein, “stage B” refers to dogs with a heart murmur due to mitral regurgitation but without clinical signs of congestive heart failure. “Stage B” includes stage B1 (absence of cardiac remodeling and stage B2 (presence of cardiac remodeling).

As used herein, stage C″ refer to dogs having congestive heart failure.

As used herein, “degenerative mitral valve disease,” “DMVD,” “chronic valvular disease,” “CVD,” “myxomatous mitral valve disease,” and “MMVD” can be used interchangeably and refers to progressive valvular degeneration that causes mitral regurgitation and/or congestive heart failure (CHF) and includes stage A, stage B, and stage

C.

As used herein, “dysbiosis index” or “DI” is quantified by as a single numerical value that measured the closeness (l₂-norm) of the test sample to the mean (prototype) of each class as disclosed in “A Dysbiosis Index to Assess Microbial Changes in Fecal Samples of Dogs with Chronic Inflammatory Enteropathy” by AlShawaqfeh et al, FEMS Microbiology Ecology, vol. 93, no. 11, pp 1-8 (2017) (doi:0.1093/femsec/fix136). As discussed in the above article, DI is defined as the difference between (Euclidean distance between the test sample and the healthy class centroid) and the (Euclidean distance between the test and the diseased class centroid). DI is calculated mathematically as follows: the DI of a text sample z is defined as:

DI(z;μ_{C}_{_{D}}, μ_{C}_{_{H}})=∥z−μ_{C}_{_{H}}∥₂−∥z−μ_{C}_{_{D}}∥₂

Where μ_{CD} and μ_{C}_{_{H}} stand for the centroid of the diseased and healthy samples in the training set, respectively.

The methods disclosed herein may lack any step that is not specifically disclosed herein. Thus, a disclosure of an embodiment using the term “comprising” includes a disclosure of embodiments “consisting essentially of” and “consisting of” the steps identified. Any embodiment disclosed herein can be combined with any other embodiment disclosed herein unless explicitly and directly stated otherwise.

Unless defined otherwise, all technical and scientific terms and any acronyms used herein have the same meanings as commonly understood by one of ordinary skill in the art in the field of the invention. Although any compositions, methods, articles of manufacture, or other means or materials similar or equivalent to those described herein can be used in the practice of the present invention, the preferred compositions, methods, articles of manufacture, or other means or materials are described herein.

All patents, patent applications, publications, and other references cited or referred to herein are incorporated herein by reference to the extent allowed by law. The discussion of those references is intended merely to summarize the assertions made therein. No admission is made that any such patents, patent applications, publications or references, or any portion thereof, are relevant prior art for the present invention and the right to challenge the accuracy and pertinence of such patents, patent applications, publications, and other references is specifically reserved.

Embodiments

The present inventor has discovered that heart disease can be diagnosed based on specific microbiome including a dysbiosis index as well as specific genera and species. Further, the inventor has discovered that the present microbiome can be used to diagnose early stage degenerative mitral valve disease. Such methods allow for an inexpensive and efficient diagnosis for conditions that can be difficult to diagnose as well as costly.

In one embodiment, a method of diagnosing heart disease in a canine can comprise measuring a normalized relative abundance of fecal bacteria including Faecalibacterium, Turicibacter, Streptococcus, E. Coli, Blautia, Fusobacterium, and C. hiranonis, calculating a dysbiosis index based on the fecal bacteria, and determining that the canine has heart disease if the dysbiosis index is greater than −1.0.

Generally, the canine can be diagnosed with DMVD if the dysbiosis index is greater than −1.0. However, in one aspect, the determining step can also include determining that the canine has early stage DMVD when the dysbiosis index is between −1 and 0.5. Additionally, in another aspect, the present methods can further determine that the canine has early stage DMVD B1 when the dysbiosis index is between −1 and −0.25. In still another aspect, the present methods can further determine that the canine has early stage DMVD B2 when the dysbiosis index is between −0.25 and 0.5. In yet another aspect, the present methods can further determine that the canine has congestive heart failure when the dysbiosis index is greater than 0.5.

In another embodiment, a method of diagnosing early stage DMVD in a canine can comprise measuring a normalized relative abundance of a biomarker selected from the group consisting of Catenibacterium mitsuokai, Butyricicoccus pullicaecorum, Bacteroides coprocola, Bacteroides plebeius, Allobaculum stercoricanis, or combinations thereof; and determining that the canine has early stage DMVD if the normalized relative abundance of Catenibacterium mitsuokai is between 0.3 to 3, the normalized relative abundance of Butyricicoccus pullicaecorum is between 0.14 to 0.35, the normalized relative abundance of Bacteroides coprocola is between 0.6 to 1.3, the normalized relative abundance of Bacteroides plebeius is between 0.1 to 0.8, or the normalized relative abundance of Allobaculum stercoricanisi is between 0.1 and 1.5.

Generally, the canine can be diagnosed with early stage DMVD using various biomarkers, i.e., bacteria, discussed herein having specific normalized relative abundances. However, in one aspect, the diagnosis can be based at least two biomarkers. In another aspect, the diagnosis can be based on at least three biomarkers. In still another aspect, the diagnosis can be based on at least four biomarkers. In yet another aspect, the diagnosis can be based on all five biomarkers.

In yet another embodiment, a method of diagnosing early stage DMVD in a canine can comprise measuring a normalized relative abundance of bacteria in a genus, wherein the genus is selected from the group consisting of Catenibacterium, Prevotella, Butyricicoccus, Faecalibacterium, Clostridium, Allobaculum, or combinations thereof; and determining that the canine has early stage DMVD if the normalized relative abundance of the bacteria in the Catenibacterium genus is between 0.3 to 3, the normalized relative abundance of the bacteria in the Prevotella genus is between 0.5 to 4, the normalized relative abundance of the bacteria in the Butyricicoccus genus is between 0.14 to 0.4, the normalized relative abundance of the bacteria in the Faecalibacterium genus is between 0.012 to 0.04, the normalized relative abundance of the bacteria in the Clostridium genus is between 2 and 4, or the normalized relative abundance of the bacteria in the Allobaculum genus is between 0.2 and 1.5.

Generally, the canine can be diagnosed with early stage DMVD using bacteria in various genera, discussed herein having specific normalized relative abundances. However, in one aspect, the diagnosis can be based at least two genera. In another aspect, the diagnosis can be based on at least three genera. In still another aspect, the diagnosis can be based on at least four genera. In another aspect, the diagnosis can be based on at least five genera. In yet another aspect, the diagnosis can be based on all six biomarkers.

### EXAMPLES

The following non-limiting examples are illustrative of embodiments of the present disclosure.

Example 1-DMVD Study of Canines

Clinically healthy dogs 7 years of age or older without a heart murmur and without concurrent systemic disease were prospectively enrolled as controls (group A, N=29). A cohort of dogs 7 years of age or older with a left apical systolic murmur, echocardiographic (echo) diagnosis of thickened and prolapsing mitral valve leaflet(s) and mitral regurgitation, as well as clinical history and physical exam consistent with stage B1, B2, C, or D DMVD were considered for group B1 (N=34), group B2 (N=25), and group C/D (N=25), respectively. Any dog with severe concurrent systemic disease including diabetes, cancer, or renal failure, or those with any congenital heart disease were excluded. Dogs with signs of gastrointestinal illness such as vomiting or diarrhea and those that had received antibiotics within 30 days were also excluded. Fecal samples were obtained from these dogs.

Diet intervention study with preclinical DMVD dogs

Dogs with preclinical DMVD and with body weight less than 15 kilograms were considered for inclusion in the study. Dogs were randomly assigned to two diet groups, control diet (CON, N=9) and diet supplemented with cardiac protect blend (CPB, N=10). The two diets were previously described in details. Dog were fed their assigned diets as their sole source of nutrition for 6 months. Clinical measures and fecal samples were taken at baseline, 3 and 6 months.

Fecal DNA extraction and metagenomic sequencing

Fecal genomic DNA (Input-450-600 ng) was fragmented on the Covaris LE220 instrument targeting 375 bp inserts. Automated Illumina libraries were constructed with the KAPA Hyper PCR-free library prep kit (KAPA Biosystems/Roche) on the SciClone NGS platform (Perkin Elmer). The fragmented genomic DNA was size selected on the SciClone instrument with AMPure XP beads to tighten the distribution of DNA fragments to ensure the average insert of the libraries were between 350-375 bps. The manufacturer's protocol as provided by Perkin Elmer was followed, with the following exception: Post ligation, the libraries were purified twice with a 0.7× AMPure bead/sample ratio to eliminate any residual adaptors. An aliquot of the final libraries were diluted 1:5 and quantitated on the Caliper GX instrument (Perkin Elmer). The concentration of each library was accurately determined through qPCR utilizing the KAPA library Quantification Kit according to the manufacturer's protocol (KAPA Biosystems/Roche) to produce cluster counts appropriate for Illumina NovaSeq6000 instrument. Libraries were pooled and run over 0.1 of a NovaSeq6000 S4 flow cell using the XP workflow and running a 150×10×10×150 sequencing recipe in accordance with manufacturer's protocol. Approximately 5Gb paired-end sequences were generated for each sample.

Dysbiosis index

The abundances of eight bacterial groups, including total bacteria, Faecalibacterium, Turicibacter, Escherichia coli, Streptococcus, Blautia, Fusobacterium and Clostridium hiranonis, were evaluated on the fecal DNA samples from the four groups of DMVD dogs: groups A (N=31), B1 (N=35), B2 (N=25), and C/D (N=30). The qPCR primer sets, protocol and the method for dysbiosis index (DI) were described previously in

AlShawaqfeh et al. FEMS Microbiology Ecology, vol. 93, no. 11, pp 1-8 (2017). A negative DI indicates normobiosis, whereas a positive DI indicates dysbiosis. The reference interval for C. hiranonis, a beneficial bacterium, was between 5.1-7.1.

Bioinformatics analysis

Quality of the sequences were examined using fastQC. Paired-end sequences were stitched together using PEAR with default settings. Trimmomatic was used to remove low quality sequences while Bowtie2 was run to screen out contaminant sequences mapped to the canine (CanFam3.1) or PhiX reference genomes. Low quality sequences were scanned with a 4-base wide sliding window and removed if the average quality score per base dropped below 20. Sequences with less than 50 bases were also removed. Phylogenetic analysis was performed using MetaPhlAn 2.0 and the abundance of UniRef90 gene families and MetaCyc pathways was calculated using HUMAnN 2.0.

Statistical analysis

For the cross-sectional study, multi-group comparisons were performed using Kruskal-Wallis tests. Dunn's multiple comparisons were performed on the significant taxa. For the diet intervention study, changes from 3 months and 6 months over baseline were calculated for each taxon. Then the differences between group means (CPB vs. CON) were calculated. A positive number indicates an increase in CPB from CON, while a negative number indicates a decrease.

Results

Table 1 provides bacterial species with differential abundances among groups A, B1, B2, and C/D, which refer to the four stages of canine DMVD. Table 2 provides changes between CPB and CON bacterial abundance at 3 months and 6 months, which were normalized over baseline values with means calculated. Twenty-three bacterial species were changes among the four groups of DMVD dogs (Table 1). Among them, seven bacteria, B. plebeiu, A. stercoricanis, E. biforme, B. coprocola, B. pullicaecorum, C. mitsuokai, and P. copri, whose abundances were decreased with DMVD severity had increased abundances in CPB-fed dogs vs. CON-fed dogs, while the abundance of Bacteroides vulgatus was reduced in CPB-fed dogs vs. CON-fed dogs (Table 2).

TABLE 1

| | P | Mean | | | |
|---|---|---|---|---|---|
| Genus Species | value | A | B1 | B2 | C/D |
| Lactococcus lactis | 0.0008 | 0 | 0.001 | 0.003 | 0.054 |
| Streptococcus minor | 0.0019 | 0.002 | 0.002 | 0.002 | 0.033 |
| Subdoligranulum unclassified | 0.0018 | 0.014 | 0.008 | 0 | 0.049 |
| Catenibacterium mitsuokai | 0.0011 | 3.994 | 1.494 | 0.319 | 0.233 |
| Eubacterium biforme | 0.0022 | 1.299 | 0.383 | 0.047 | 0.071 |
| Prevotella copri | 0.0046 | 5.265 | 1.478 | 1.628 | 0.261 |
| Butyricicoccus pullicaecorum | 0.0042 | 0.455 | 0.256 | 0.152 | 0.134 |
| Flavonifractor plautii | 0.0075 | 0.008 | 0.001 | 0 | 0.02 |
| Bacteroides coprocola | 0.0096 | 1.925 | 0.918 | 0.811 | 0.44 |
| Bacteroides plebeius | 0.0137 | 0.934 | 0.727 | 0.277 | 0.057 |
| Lactococcus garvieae | 0.0126 | 0 | 0 | 0.002 | 0 |
| Faecalibacterium prausnitzii | 0.0148 | 0.048 | 0.013 | 0.015 | 0.01 |
| Escherichia coli | 0.0165 | 1.381 | 2.031 | 5.269 | 2.875 |
| Escherichia unclassified | 0.0224 | 0.706 | 0.585 | 2.274 | 1.358 |
| Bacteroides vulgatus | 0.0273 | 1.077 | 1.419 | 3.774 | 0.982 |
| Parabacteroides distasonis | 0.0301 | 0 | 0.034 | 0.021 | 0.373 |
| Turicibacter unclassified | 0.0299 | 0.514 | 0 | 0 | 0 |
| Clostridium perfringens | 0.0422 | 0.087 | 0.701 | 2.659 | 2.054 |
| Allobaculum stercoricanis | 0.0429 | 1.93 | 1.095 | 0.807 | 0.063 |
| Bilophila unclassified | 0.0404 | 0 | 0 | 0.143 | 0.095 |
| Proteus mirabilis | 0.0435 | 0 | 0.008 | 0 | 0.163 |
| Streptococcus infantarius | 0.048 | 0.001 | 0.026 | 0 | 0 |
| Lachnospiraceae bacterium 7 1 | 0.048 | 0 | 0 | 0 | 0.002 |
| 58FAA | | | | | |

TABLE 2

| | 3 months - baseline | | CPB − | 6 months - baseline | | CPB − |
|---|---|---|---|---|---|---|
| Genus Species | CON | CPB | CON | CON | CPB | CON |
| Bacteroides plebeius | 0.0813 | 0.6817 | 0.6 | 0.0813 | 0.6817 | 0.6 |
| Allobaculum stercoricanis | −0.9075 | −0.1596 | 0.7 | −2.4208 | −1.123 | 1.3 |
| Eubacterium biforme | −1.3652 | −0.4872 | 0.9 | −1.4233 | −0.6205 | 0.8 |
| Bacteroides coprocola | 0.0018 | 0.712 | 0.7 | 0.5066 | 2.7123 | 2.2 |
| Butyricicoccus pullicaecorum | 0.0616 | 0.0184 | 0 | −0.1575 | −0.0084 | 0.1 |
| Catenibacterium mitsuokai | −3.5703 | −0.2561 | 3.3 | −3.6665 | −1.179 | 2.5 |
| Prevotella copri | 0.7739 | 8.8631 | 8.1 | 12.5194 | 17.827 | 5.3 |
| Bacteroides vulgatus | 1.6525 | 0.2789 | −1.4 | 0.5871 | 0.3112 | −0.3 |

Table 3 provides bacterial genus with differential abundances among groups A, B1, B2, and C/D, which refer to the four stages of canine DMVD. Table 3 provides changes between CPB and CON bacterial abundance at 3 months and 6 months, which were normalized over baseline values with means calculated. Three bacterial genera, Prevotella, Catenibacterium, and Allobaculum, had decreased abundances with DMVD severity (Table 3), but their abundances were increased in dogs fed CPB diet vs. CON diet (Table 4).

TABLE 3

| Genus | P value | A | B1 | B2 | C/D |
|---|---|---|---|---|---|
| Subdoligranulum | 0.0018 | 0.014 | 0.008 | 0 | 0.049 |
| Catenibacterium | 0.0011 | 3.994 | 1.494 | 0.319 | 0.233 |
| Prevotella | 0.0046 | 5.265 | 1.478 | 1.628 | 0.261 |
| Lactococcus | 0.0031 | 0 | 0.001 | 0.005 | 0.054 |
| Butyricicoccus | 0.0042 | 0.455 | 0.256 | 0.152 | 0.134 |
| Flavonifractor | 0.0075 | 0.008 | 0.001 | 0 | 0.02 |
| Escherichia | 0.0116 | 2.086 | 2.616 | 7.542 | 4.233 |
| Faecalibacterium | 0.0148 | 0.048 | 0.013 | 0.015 | 0.01 |
| Clostridium | 0.0388 | 1.768 | 2.692 | 3.832 | 4.834 |
| Allobaculum | 0.0429 | 1.93 | 1.095 | 0.807 | 0.063 |
| Bilophila | 0.0404 | 0 | 0 | 0.163 | 0.096 |
| Proteus | 0.0435 | 0 | 0.008 | 0 | 0.163 |

TABLE 4

| | 3 months - baseline | | CPB − | 6 months - baseline | | CPB − |
|---|---|---|---|---|---|---|
| Genus | CON | CPB | CON | CON | CPB | CON |
| Prevotella | 0.7739 | 8.8631 | 8.1 | 12.5194 | 17.827 | 5.3 |
| Catenibacterium | −3.5703 | −0.2561 | 3.3 | −3.6665 | −1.179 | 2.5 |
| Allobaculum | −0.9075 | −0.1596 | 0.7 | −2.4208 | −1.123 | 1.3 |

The DI (log DNA/gram of feces) for group A healthy dogs was −1.48, but was increased to −0.6, −0.07 in groups B1 and B2 preclinical DMVD dogs respectively, and to 1.47 in group C/D dogs with congestive heart failure. Thus, the DI can be used as an early indicator of DMVD in dogs (Table 5). In addition, the abundance of Clostridium hiranonis, a beneficial bacterial that converts primary bile acids to secondary bile acids, was within the reference interval in healthy dogs, but outside the interval in dogs with DMVD (Table 5).

TABLE 5

| | Mean (log DNA/gram of feces) | | | | |
|---|---|---|---|---|---|
| | A | B1 | B2 | C/D | Reference interval |
| Dysbiosis Index | −1.48 | −0.6 | −0.07 | 1.47 | <0 normobiosis; >0 |
| | | | | | dysbiosis |
| Total bacteria | 11.28 | 11.33 | 11.31 | 11.24 | 10.6-11.4 |
| Faecalibacterium | 6.05 | 5.39 | 5.14 | 5.1 | 3.4-8.0 |
| Turicibacter | 7.18 | 6.74 | 6.92 | 6.4 | 4.6-8.1 |
| Streptococcus | 6.13 | 6.43 | 5.83 | 6.55 | 1.9-8.0 |
| E. Coli | 4.39 | 4.43 | 5.67 | 5.64 | 0.9-8.0 |
| Blautia | 11.03 | 11.35 | 11.23 | 11.32 | 9.5-11.0 |
| Fusobacterium | 8.7 | 8.61 | 8.88 | 8.18 | 7.0-10.3 |
| C. hiranonis | 5.12 | 5.06 | 4.43 | 3.9 | 5.1-7.1 |

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method of diagnosing heart disease in a canine, comprising:
measuring a normalized relative abundance of fecal bacteria including Faecalibacterium, Turicibacter, Streptococcus, E. Coli, Blautia, Fusobacterium, and C. hiranonis;
calculating a dysbiosis index based on the fecal bacteria; and
determining that the canine has heart disease if the dysbiosis index is greater than −1.0.

2. The method of claim 1, further determining that the canine has early stage degenerative mitral valve disease when the dysbiosis index is between −1 and 0.5.

3. The method of claim 2, further determining that the early stage degenerative mitral valve disease is B1 when the dysbiosis index is between −1 and −0.25.

4. The method of claim 2, further determining that the early stage degenerative mitral valve disease is B2 when the dysbiosis index is between −0.25 and 0.5.

5. The method of claim 1, further determining that the canine has congestive heart failure when the dysbiosis index is greater than 0.5.

6. A method of diagnosing early stage degenerative mitral valve disease in a canine, comprising:
measuring a normalized relative abundance of a biomarker selected from the group consisting of Catenibacterium mitsuokai, Butyricicoccus pulhcaecorum, Bacteroides coprocola, Bacteroides plebeius, Allobaculum stercoricanis, or combinations thereof; and
determining that the canine has early stage degenerative mitral valve disease if the normalized relative abundance of Catenibacterium mitsuokai is between 0.3 to 3, the normalized relative abundance of Butyricicoccus pulhcaecorum is between 0.14 to 0.35, the normalized relative abundance of Bacteroides coprocola is between 0.6 to 1.3, the normalized relative abundance of Bacteroides plebeius is between 0.1 to 0.8, or the normalized relative abundance of Allobaculum stercoricanisi is between 0.1 and 1.5.

7. The method of claim 6, wherein the determining is based on at least two biomarkers.

8. The method of claim 6, wherein the determining is based on at least three biomarkers.

9. The method of claim 6, wherein the determining is based on at least four biomarkers.

10. A method of diagnosing early stage degenerative mitral valve disease in a canine, comprising:
measuring a normalized relative abundance of bacteria in a genus, wherein the genus is selected from the group consisting of Catenibacterium, Prevotella, Butyricicoccus, Faecalibacterium, Clostridium, Allobaculum, or combinations thereof; and
determining that the canine has early stage degenerative mitral valve disease if the normalized relative abundance of the bacteria in the Catenibacterium genus is between 0.3 to 3, the normalized relative abundance of the bacteria in the Prevotella genus is between 0.5 to 4, the normalized relative abundance of the bacteria in the Butyricicoccus genus is between 0.14 to 0.4, the normalized relative abundance of the bacteria in the Faecalibacterium genus is between 0.012 to 0.04, the normalized relative abundance of the bacteria in the Clostridium genus is between 2 and 4, or the normalized relative abundance of the bacteria in the Allobaculum genus is between 0.2 and 1.5.

11. The method of claim 10, wherein the determining is based on at least two genera.

12. The method of claim 10, wherein the determining is based on at least three genera.

13. The method of claim 10, wherein the determining is based on at least four genera.
